(19) United States
(12) Patent Application Publication
(10) Pub. No.: US 2023/0204563 A1
(43) Pub. Date: Jun. 29, 2023

(54) SENSING DEVICE AND SENSING METHOD

(71) Applicant: National Tsing Hua University, Hsinchu City (TW)

(72) Inventors: Yu-Lin Wang, Hsinchu City (TW); Shu-Yi Tsai, Hsinchu City (TW)

(73) Assignee: National Tsing Hua University, Hsinchu City (TW)

(21) Appl. No.: 17/584,397

(22) Filed: Jan. 26, 2022

(30) Foreign Application Priority Data
Dec. 23, 2021 (TW) 110148521

(51) Int. Cl. G01N 33/487; G01N 33/543

(52) U.S. Cl. CPC G01N 33/48728; G01N 33/5438; G01N 27/4145

(57) ABSTRACT

A sensing device is provided. The sensing device includes a transistor, a disposable electrode, and a remote electrode. The transistor includes an extended gate, source and drain. The remote electrode is configured to receive a reference voltage. The disposable electrode is coupled between the transistor and the remote electrode. The disposable electrode includes a proximal end and a distal end. The proximal end of the disposable electrode is coupled to the extended gate of the transistor. The distal end of the disposable electrode is coupled to the remote electrode. The disposable electrode is adapted to load a cell and receive a membrane potential of the cell. The disposable electrode provides a gate voltage to the extended gate based on the change of the membrane potential and the reference voltage. The transistor provides different transistor currents at the drain based on the change of the gate voltage.

### CROSS-REFERENCE TO RELATED APPLICATION

This application claims the priority benefit of Taiwan application serial no. 110148521, filed on Dec. 23, 2021. The entirety of each of the above-mentioned patent applications is hereby incorporated by reference herein and made a part of this specification.

### BACKGROUND

### Technical Field

The disclosure relates to a sensing device, and in particular to a sensing device and a sensing method.

### Description of Related Art

As a measurement system for drug screening currently on the market, it can be divided into several major steps: target selection, lead drug discovery, medicinal chemistry, pharmacological research, and candidate development drugs. In the step of pharmacological research, animal experiments or cell experiments are currently used to verify the experimental results. In cell experiments, a large number of cells and drugs are required to perform optical responses to measure. Due to the long incubation time required for a large number of cells, some cells may have been apoptotic by the time the optical response was measured.

In addition, if the invasive measurement is adopted for the cells, the survival time of the cells after the measurement is short, and it is difficult to perform other measurements again.

In addition, in order to prevent cross-contamination, used instruments need to be cleaned or replaced in bulk, which increases both time and economic costs.

### SUMMARY

The present application provides a sensing device and a sensing method, which can measure cells with only a small number of cells.

The sensing device of the present application includes a transistor, a disposable electrode, and a remote electrode. The transistor includes an extended gate, source and drain. The remote electrode is configured to receive a reference voltage. The disposable electrode is coupled between the transistor and the remote electrode. The disposable electrode includes a proximal end and a distal end. The proximal end of the disposable electrode is coupled to the extended gate of the transistor. The distal end of the disposable electrode is coupled to the remote electrode. The disposable electrode is adapted to load a cell and receive a membrane potential of the cell. The disposable electrode provides a gate voltage to the extended gate based on the change of the membrane potential and the reference voltage. The transistor provides different transistor currents at the drain based on the change of the gate voltage.

The sensing method of the present application includes the following steps: receiving a reference voltage by a remote electrode; loading a cell by a disposable electrode; providing a gate voltage to an extended gate based on the change of the membrane potential and the reference voltage by the disposable electrode; and providing different transistor currents based on the change of the gate voltage by the transistor.

Based on the above, the sensing device and the sensing method of the present application only need a small number of cells to measure cells.

In order to make the above-mentioned features and advantages of the present application more obvious and easier to understand, the following specific examples are given, and are described in detail as follows in conjunction with the accompanying drawings.

### BRIEF DESCRIPTION OF THE DRAWINGS

FIG. 1 is a schematic diagram of a sensing device according to an embodiment of the present application.

FIG. 2A is a schematic diagram of measuring cells by a sensing device according to an embodiment of the present application.

FIG. 2B is a schematic diagram of measuring the response of a drug to the cell by a sensing device according to an embodiment of the present application.

FIG. 3A is a schematic diagram of the effect of drug concentration on current intensity according to an embodiment of the present application.

FIG. 3B is a schematic diagram of the effect of ion concentration on current intensity according to an embodiment of the present application.

FIG. 4 is a schematic diagram of a circuit package according to an embodiment of the present application.

FIG. 5 is a flow chart of a sensing method according to an embodiment of the present application.

### DESCRIPTION OF THE EMBODIMENTS

In order to make the content of the present application more comprehensible, the following specific embodiments are given as examples according to which the present application can indeed be implemented. In addition, wherever possible, elements/components/steps using the same reference numerals in the drawings and embodiments represent the same or similar parts.

And, unless defined otherwise, all terms (including technical and scientific terms) used herein have the same meaning as commonly understood by one of ordinary skill in the art to which the application belongs. It will be further understood that terms such as those defined in commonly used dictionaries should be construed to have meanings consistent with their meanings in the context of the related art and the present application. And it is not to be construed in an idealized or overly formal sense unless explicitly defined as such herein.

The present application can be understood by reference to the following detailed description taken in conjunction with the accompanying drawings. It should be noted that, for the sake of easy understanding of the reader and the simplicity of the drawings, the drawings in the present disclosure only depict a part of the electronic device, and specific elements in the drawings are not drawn according to actual scale. In addition, the number and size of each element in the figures are for illustration only, and are not intended to limit the scope of the present application.

It should be noted that the following examples can replace, reorganize, and mix the technical features in several different embodiments to complete other embodiments without departing from the spirit of the present application. Moreover, in the following description and claims, words such as “comprising” and “including” are open-ended words, so they should be interpreted as meaning “including but not limited to...”.

Generally speaking, the potential inside the cell membrane of a cell is lower than the potential on the surface of the cell membrane. That is to say, cells are in a polarized state under normal circumstances. And, as the function of the cell changes, the polarization state of the cell may change accordingly. In other words, by measuring the change in the potential of the cell membrane surface (also called the cell membrane potential), it can be known whether the function of the cell works normally.

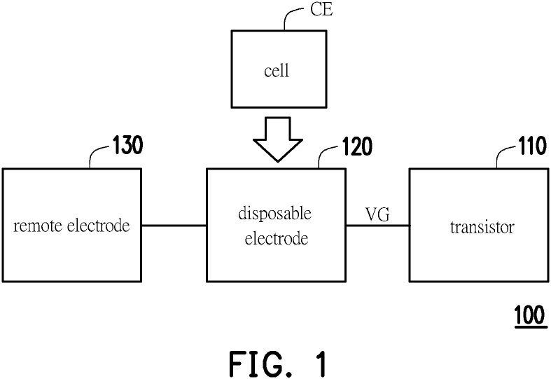

FIG. 1 is a schematic diagram of a sensing device according to an embodiment of the present application. Referring to FIG. 1, a sensing device 100 includes a transistor 110, a disposable electrode 120 and a remote electrode 130. The transistor 110 includes an extended gate, source and drain. The remote electrode 130 is configured to receive a reference voltage. The disposable electrode 120 is coupled between the transistor 110 and the remote electrode 130. The disposable electrode 120 includes a proximal end and a distal end. The proximal end of the disposable electrode 120 is coupled to the extended gate of the transistor 110. The distal end of the disposable electrode 120 is coupled to the remote electrode 130. The disposable electrode 120 is adapted to load a cell CE, the disposable electrode 120 is adapted to receive a membrane potential of the cell CE. The disposable electrode 120 provides a gate voltage VG to the extended gate of the transistor 110 based on the change of the membrane potential and the reference voltage. The transistor 110 provides different transistor currents at the drain based on the change of the gate voltage VG.

In this way, through the measured different transistor currents, the change of the cell membrane potential of the cell CE can be known, and then it can be determined whether the function of the cell CE is functioning normally.

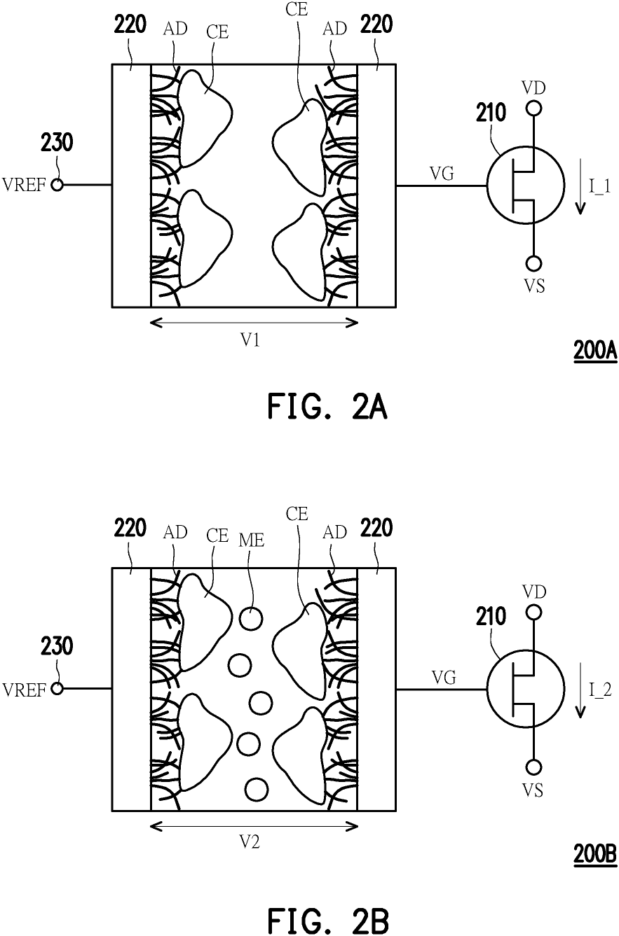

FIG. 2A is a schematic diagram of measuring cells by a sensing device according to an embodiment of the present application. Referring to FIG. 1 and FIG. 2A, the sensing device 200A of FIG. 2A is an embodiment of FIG. 1A, but the present application is not limited thereto. The sensing device 200A includes a transistor 210, a disposable electrode 220 and a remote electrode 230.

In this embodiment, the transistor 210 may include an extended gate, a source and a drain. The extended gate is configured to receive a gate voltage VG. The source is used to receive the source voltage VS. The drain is used to receive the drain voltage VD. And, the transistor 210 provides transistor current I_1 at the drain based on the gate voltage VG.

In this embodiment, the transistor 210, the disposable electrode 220 and the remote electrode 230 form a Stretch-Out Electrical Double Layer-Gated (EDL-gated) Field Effect Transistor (FET), but the present application is not limited thereto. Specifically, the disposable electrode 220 includes proximal and distal double-layer electrodes, and the double-layer electrodes form an electrical double-layer capacitor. Also, the electrode at the proximal end of the disposable electrode 220 is coupled to the extended electrode of the transistor 210. The electrode at the distal end of the disposable electrode 220 is coupled to the remote electrode 230. In addition, the remote electrode 230 can be used to receive the reference voltage VREF.

In this embodiment, the cell CE can be arranged in a reagent, and the reagent is placed on the disposable electrode 220. Also, the electrodes at the proximal end and the electrodes at the distal end of the disposable electrode 220 may be provided with adhesive layers AD, respectively. Next, the cell CE can be loaded onto the disposable electrode 220 by placing the reagent on the disposable electrode 220. In other words, the disposable electrode 220 can load the cell CE with the adhesive layer AD. In one embodiment, the adhesive layer AD includes one of fibronectin and gelatin, but the present application is not limited thereto.

It should be noted that, before and after the disposable electrode 220 is provided with the adhesive layer AD, the transistor current I_1 can be measured separately to obtain a reference value of the transistor current I_1. Specifically, when different adhesive layers AD is provided, the measured transistor current I_1 may be different. Next, the measured transistor current I_1 is used as a reference value to perform zero correction. In this way, subsequent measurements can obtain more accurate results.

In this embodiment, after the disposable electrode 220 loads the cell CE, the capacitance value between the double-layer electrodes of the disposable electrode 220 will change due to the influence of the cell membrane potential of the cell CE. In addition, the change of the capacitance value between the double-layer electrodes causes the first voltage V1 of the voltage difference between the double-layer electrodes to change, thereby changing the gate voltage VG. In other words, the disposable electrode 220 can provide the gate voltage VG to the extended electrode of the transistor 210 based on the change of the cell membrane potential and the reference voltage VREF. Next, the transistor 210 can provide different transistor currents I_1 at the drain of the transistor 210 based on the change of the gate voltage VG.

In this way, through the measured different transistor currents I_1, the change of the cell membrane potential of the cell CE can be known, and then it can be determined whether the function of the cell CE is functioning normally. In addition, the sensing device 100 can perform the measurement of cell CE with only a small number of cell CE. Furthermore, since the disposable electrode 120 does not use the invasive measurement on the cell CE, the cell CE after the measurement can be used for other measurements.

FIG. 2B is a schematic diagram of measuring the response of a drug to the cell by a sensing device according to an embodiment of the present application. Referring to FIG. 1 to FIG. 2B, the difference between FIG. 2B and FIG. 2A is that the sensing device 200B is further used to measure the response of the drug ME to the cell CE.

In this embodiment, in addition to the cell CE disposed in the reagent, the reagent further includes a drug ME that acts on the cell CE. After the drug ME acts on the cell CE, the cell membrane potential of the cell CE may change. Also, the second voltage V2 of the voltage difference between the double-layer electrodes of the disposable electrode 220 may change accordingly, thereby changing the gate voltage VG. Next, the transistor 210 can provide different transistor currents I_2 at the drain of the transistor 210 based on the change of the gate voltage VG.

In this way, through the measured different transistor currents I_1, the change of the cell membrane potential of the cell CE can be known, and then the response of the drug ME to the cell CE can be determined. In other words, the sensing device 200B only needs a small amount of cell CE to perform the screening of the drug ME.

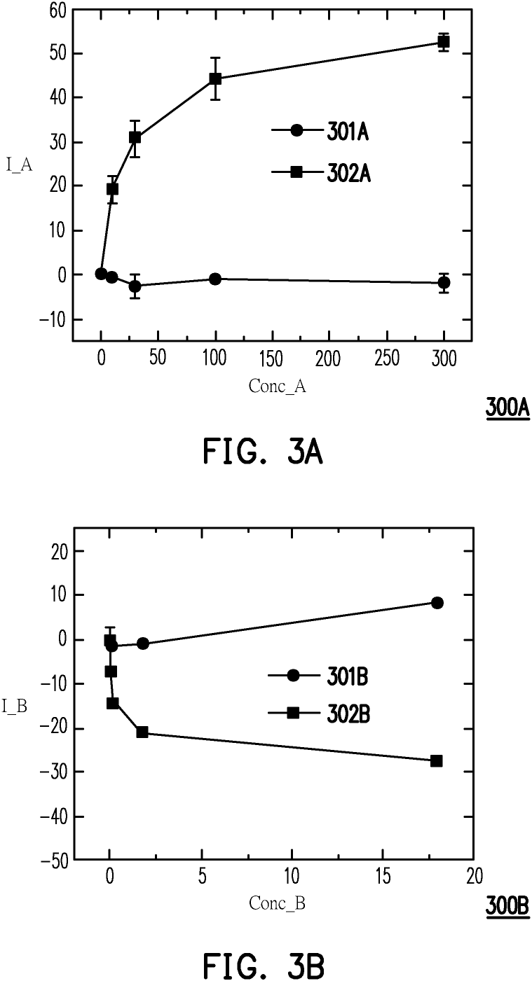

FIG. 3A is a schematic diagram of the effect of drug concentration on current intensity according to an embodiment of the present application. Referring to FIG. 1 to FIG. 3A, the vertical axis is the current I_A, and the horizontal axis is the concentration Conc_A of the drug ME in the current intensity graph 300A. The unit of current I_A is microampere (uA) and the unit of concentration Conc_A is nanomol per liter (nM). The current I_A represents the difference between the value of the transistor current I_2 and the value of the transistor current I_1 at the concentration Conc_A of the drug ME. In other words, the value of the transistor current I_2 can be obtained by adding the value of the transistor current I_1 to the value of the transistor current I_1. That is, the transistor current I_1 is a reference value, the transistor current I_2 is a measured value, and the current I_A is a variable value. In addition, line 301A represents the value of current I_A measured when the disposable electrode 220 is not loaded with cells CE. Line 302A represents the value of current I_A measured when the disposable electrode 220 is loaded with cells CE.

In this embodiment, as the concentration Conc_A of the drug ME increases, on line 301A, the value of the current I_A measured when the cell CE is not yet loaded remains almost unchanged. However, as the concentration Conc_A of the drug ME increases, on line 302A, the value of the current I_A measured while loading the cells CE increases gradually. That is to say, the change of the current I_A is indeed caused by the cell CE, not simply caused by the change of the concentration Conc_A of the drug ME.

In one embodiment, the cell CE can be the H9c2 cell in the cardiomyocytes, and the drug ME can be the drug Nifedipine, but the present application is not limited thereto.

In this way, through the measured different currents I_A, the change of the cell membrane potential of the drug ME to the cell CE can be known, and then the response of the drug ME to the cell CE can be determined.

FIG. 3B is a schematic diagram of the effect of ion concentration on current intensity according to an embodiment of the present application. Referring to FIG. 1 to FIG. 3B, the vertical axis is the current I_B, and the horizontal axis is the concentration Conc_B of the ion in the current intensity graph 300B. The unit of current I_B is uA and the unit of concentration Conc_B is nM. The current I_B represents the difference between the value of the transistor current I_2 and the value of the transistor current I_1 at the concentration Conc_B of the ion. In other words, the value of the transistor current I_2 can be obtained by adding the value of the transistor current I_1 to the value of the transistor current I_1. That is, the transistor current I_1 is a reference value, the transistor current I_2 is a measured value, and the current I_B is a variable value. In addition, line 301B represents the value of current I_B measured when the disposable electrode 220 is not loaded with cells CE. Line 302B represents the value of current I_B measured when the disposable electrode 220 is loaded with cells CE.

In this embodiment, as the concentration Conc_B of the ion increases, on line 301B, the value of the current I_B measured when the cell CE is not yet loaded only increases slightly. However, as the concentration Conc_B of the ion increases, on line 302B, the value of the current I_B measured while loading the cells CE reduces significantly. That is to say, the change of the current I_B is indeed caused by the cell CE, not simply caused by the change of the concentration Conc_B of the ion.

In one embodiment, the cells CE can be H9c2 cells in cardiomyocytes, the ions are calcium ions (Ca2+), and the drug ME can be the drug Nifedipine, but the application is not limited thereto. When the drug Nifedipine acts on H9c2 cells, part of the Ca2+ channel of H9c2 cells may be blocked, resulting in the change of the concentration Conc_B of Ca2+. That is, the response of H9c2 cells to the drug Nifedipine can be known by measuring the current I_B.

In this way, through the measured different currents I_B, the change of the cell membrane potential of the drug ME on the cell CE can be known, and then the response of the drug ME to the cell CE can be determined.

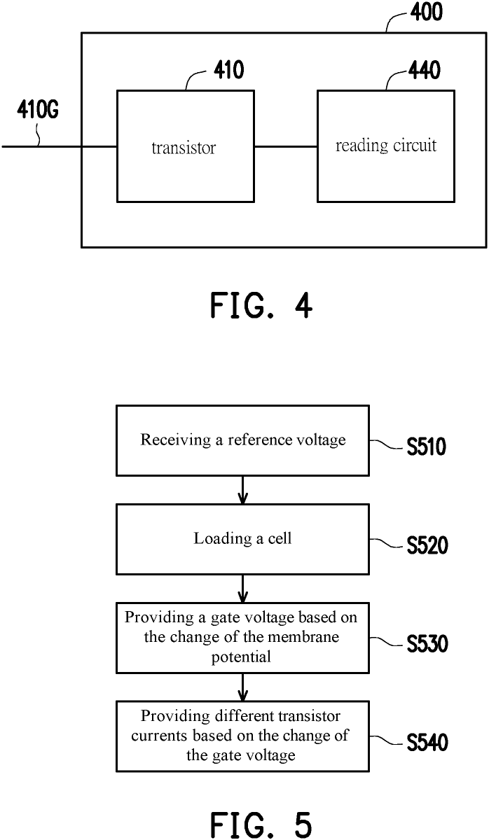

FIG. 4 is a schematic diagram of a circuit package according to an embodiment of the present application. Referring to FIG. 1, FIG. 2A, FIG. 2B and FIG. 4, the sensing device 100 further includes a reading circuit 440. The reading circuit 440 is coupled to the transistor 410. For details of the transistor 410, reference may be made to the description of the transistor 110 in FIG. 1, and details are not repeated here.

In this embodiment, the reading circuit 440 can receive the transistor current I_1 or the transistor current I_2 from the transistor 410. In addition, the reading circuit 440 can determine the state of the cell CE according to the transistor current I_1 or the transistor current I_2. In one embodiment, the reading circuit 440 can determine the polarization state of the cell CE according to the transistor current I_1 or the transistor current I_2. For example, the cell CE can include cardiomyocytes. The reading circuit 440 can determine the state of the ion channel (e.g., Ca+ channel) of the cardiomyocyte according to the transistor current I_1 or the transistor current I_2.

In this way, through the measured transistor current I_1 or the transistor current I_2, the reading circuit 440 can determine the polarization state of the cell CE, and then determine whether the function of the cell CE operates normally.

In one embodiment, the remote electrode 130 and the disposable electrode 120 may be integrally provided in a replaceable disposable package. In other words, the disposable package is replaceably coupled to the extended gate 410G of the transistor 110. Moreover, each time the cell CE is measured, only the disposable electrode 120 of the disposable package is in contact with the cell CE, and the transistor 110 may not be in contact with the cell CE. In other words, after each measurement of the cell CE, only the remote electrode 130 and the disposable electrode 120 of the disposable package need to be replaced, but the transistor 410 can continue to be used.

It should be noted that the measurement of the cell membrane potential of the cell CE by the disposable electrode 120 adopts the concept of relative potential rather than the concept of absolute potential. If the concept of absolute potential is adopted, the material of the electrode for the cell CE to stay to measure the cell membrane potential and the material of the electrode for providing the reference voltage VREF will be limited. In one embodiment, the material of the electrode for the cell CE to stay is gold, and the electrode for providing the reference voltage VREF is an Ag/AgCl electrode or a Hg/HgCl2 electrode. When the two electrodes are made of different materials, the process design is more complicated.

However, when the concept of relative potential is adopted, only the voltage difference between the electrode (the remote electrode 130) providing the reference voltage VREF and the extended gate of the transistor 110 needs to be measured. Therefore, the material of the remote electrode 130 for providing the reference voltage VREF and the disposable electrode 120 for the cell CE to stay can be the same. In one embodiment, the remote electrode 130 and the disposable electrode 120 are both made of gold. In this way, the design of the manufacturing process of the remote electrode 130 and the disposable electrode 120 is relatively easy.

In addition, since the transistor 410 does not need to be replaced every time after the cell CE is measured, the transistor 410 can be permanently combined with the back-end circuit. In this embodiment, the reading circuit 440 and the transistor 410 can be disposed in the circuit package 400, and the extended gate 410G of the transistor 410 can be disposed on the boundary of the circuit package 400. In one embodiment, the circuit package 400 may include a printed circuit board (PCB), but the application is not limited thereto. For example, the reading circuit 440 and the transistor 410 may be disposed on the same PCB. In this way, the manufacturing cost of the sensing device 100 is reduced, and the maintenance is relatively easy.

Furthermore, the remote electrode 130 may include a first remote electrode and a second remote electrode. The disposable electrode 120 may include a first disposable electrode and a second disposable electrode. The first remote electrode is coupled to the first disposable electrode. The second remote electrode is coupled to the second disposable electrode. The transistor 410 is switchably coupled to one of the first disposable electrode and the second disposable electrode. For example, the transistor 410 may be coupled to the first disposable electrode and the second disposable electrode via a switch circuit. That is, the transistor 410 can receive signals from one of the first disposable electrode and the second disposable electrode in multiple ways, so as to determine the state of the cell CE. In this way, the manufacturing cost of the sensing device 100 can be further reduced.

It should be noted that the extended gate 410G of the transistor 410 in FIG. 4 is disposed on the boundary of the circuit package 400 in a partially protruding manner, but the present application is not limited thereto. The partially protruding design is to more easily couple the extended gate 410G to the proximal end of the disposable electrode 120. However, according to design requirements, the extended gate 410G may be flush with the boundary of the circuit package 400, or the extended gate 410G may be retracted within the boundary of the circuit package 400.

FIG. 5 is a flow chart of a sensing method according to an embodiment of the present application. Referring to FIG. 2A and FIG. 5, the sensing method includes Step S510, Step S520, Step S530 and Step S540. In Step S510, the reference voltage VREF is received by the remote electrode 230. In Step S520, the cell CE is loaded by the disposable electrode 220. In Step S530, the gate voltage VG is provided to the extended gate of the transistor 210 based on the change of the membrane potential and the reference voltage VREF by the disposable electrode. In Step S540, different transistor currents I_1 is provided based on the change of the gate voltage VG by the transistor 210.

In this way, through the measured different transistor currents, the change of the cell membrane potential of the cell CE can be known, and then it can be determined whether the function of the cell CE works normally.

To sum up, the sensing device and the sensing method of the present application only need a small number of cells to perform cell measurement or drug screening. Furthermore, since the disposable electrode does not use invasive measurement on the cell, the cell after the measurement can be used for other measurements.

Although the present application has been disclosed as above with embodiments, it is not intended to limit the present application, any person with ordinary knowledge in the technical field, without departing from the spirit and scope of the present application, can make some changes. Therefore, the protection scope of the present application shall be determined by the scope of the claims.

What is claimed is:

1. A sensing device, comprising:
a transistor, including an extended gate, source and drain;
a remote electrode, configured to receive a reference voltage; and
a disposable electrode, coupled between the transistor and the remote electrode, the disposable electrode includes a proximal end and a distal end, the proximal end is coupled to the extended gate of the transistor, and the distal end is coupled to the remote electrode;
wherein the disposable electrode is adapted to load a cell and receive a membrane potential of the cell, the disposable electrode provides a gate voltage to the extended gate based on the change of the membrane potential and the reference voltage,
the transistor provides different transistor currents at the drain based on the change of the gate voltage.

2. The sensing device according to claim 1, wherein the sensing device further comprises a reading circuit, coupled to the transistor, the reading circuit determines a polarization state of the cell based on the transistor current.

3. The sensing device according to claim 2, wherein the cell comprises cardiomyocytes, and the reading circuit determines the state of the ion channels of the cardiomyocytes according to the transistor current.

4. The sensing device according to claim 2, wherein the reading circuit and the transistor are disposed in a circuit package, the extended gate is disposed on the boundary of the circuit package.

5. The sensing device according to claim 1, wherein the remote electrode and the disposable electrode are integrally disposed in a replaceable disposable package, the material of the remote electrode is the same as the disposable electrode.

6. The sensing device according to claim 5, wherein the material of the remote electrode and the disposable electrode are gold.

7. The sensing device according to claim 1, wherein the remote electrode comprises a first remote electrode and a second remote electrode, the disposable electrode comprises a first disposable electrode and a second disposable electrode,
wherein the first remote electrode is coupled to the first disposable electrode, the second remote electrode is coupled to the second disposable electrode,
the transistor is switchable and coupled to one of the first disposable electrode and the second disposable electrode.

8. The sensing device according to claim 1, wherein the disposable electrode loads the cells with an adhesive layer, the adhesive layer comprises one of fibronectin and gelatin.

9. The sensing device according to claim 1, wherein the cell is arranged in a reagent, and the reagent further includes a drug acting on the cell.

10. The sensing device according to claim 1, wherein the transistor, the disposable electrode and the remote electrode form a stretch-out electrical double layer-gated field effect transistor.

11. A sensing method, comprising:
receiving a reference voltage by a remote electrode;
loading a cell by a disposable electrode;
providing a gate voltage to an extended gate of a transistor based on the change of the membrane potential and the reference voltage by the disposable electrode; and
providing different transistor currents based on the change of the gate voltage by the transistor.

12. The sensing method according to claim 11, wherein a reading circuit is coupled to the transistor, the sensing method further comprises:
determining a polarization state of the cell based on the transistor current by the reading circuit.

13. The sensing method according to claim 12, wherein the cell comprises cardiomyocytes, the sensing method further comprises:
determining the state of the ion channels of the cardiomyocytes according to the transistor current by the reading circuit.

14. The sensing method according to claim 12, further includes:
disposing the reading circuit and the transistor in a circuit package; and
disposing the extended gate on the on the boundary of the circuit package.

15. The sensing method according to claim 11, further includes:
disposing the remote electrode and the disposable electrode in a replaceable disposable package integrally, wherein the material of the remote electrode is the same as the disposable electrode.

16. The sensing method according to claim 15, wherein the material of the remote electrode and the disposable electrode are gold.

17. The sensing method according to claim 11, wherein the remote electrode comprises a first remote electrode and a second remote electrode, the disposable electrode comprises a first disposable electrode and a second disposable electrode, the sensing method further comprises:
coupling the first remote electrode to the first disposable electrode, coupling the second remote electrode to the second disposable electrode; and
switchably coupling to one of the first disposable electrode and the second disposable electrode.

18. The sensing method according to claim 11, wherein the disposable electrode loads the cells with an adhesive layer, the adhesive layer comprises one of fibronectin and gelatin.

19. The sensing method according to claim 11, further includes:
arranging the cell in a reagent, wherein the reagent further includes a drug acting on the cell.

20. The sensing method according to claim 11, wherein the transistor, the disposable electrode and the remote electrode form stretch-out electrical double layer-gated field effect transistor.
